(19) United States
(12) Patent Application Publication
(10) Pub. No.: US 2017/0095613 A1
(43) Pub. Date: Apr. 6, 2017

(54) ROTATABLE END OF DOSE FEEDBACK MECHANISM

(71) Applicant: Eli Lilly and Company, Indianapolis, IN (US)

(72) Inventor: CLAUS SCHMIDT MØLLER, Fredensborg (DK)

(73) Assignee: Eli Lilly and Company, Indianapolis, IN (US)

(21) Appl. No.: 15/314,588

(22) PCT Filed: Jun. 4, 2015
(86) PCT No.: PCT/US2015/034128

§ 371 Date: Nov. 29, 2016

Related U.S. Application Data
(60) Provisional application No. 62/008,559, filed on Jun. 6, 2014.

(51) Int. Cl. A61M 5/315

(52) U.S. Cl. CPC A61M 5/3157; A61M 5/31528; A61M 5/31585; A61M 2205/581; A61M 2205/582

(57) ABSTRACT

An injection device for injecting a medicament includes a housing, a dose setting member (20) movable to set a dose to be injected, and a signal part (7). The signal part rotates from a first rotational position to a second rotational position to increase loading on a spring or spring arm when a dose is set due to rotation of the dose setting member. An internal pressure builds up in the injection device during injection which results in the signal part being captured in the second rotational position between first and second internal parts of the injection device. After the internal pressure dissipates by a sufficient amount during injection, the signal part rotates under the urging of the loaded spring from the second rotational position back to the first rotational position. A portion of the signal part moves into contact with a surface when the signal part reaches the first rotational position to produce tactile or audible feedback indicating that an end of dose condition has been reached.

### CROSS REFERENCE TO RELATED APPLICATION

This application claims priority under 35 U.S.C. §119(e) to U.S. Provisional Application No. 62/008,559 which was filed Jun. 6, 2014 and which is hereby incorporated by reference herein.

### FIELD OF THE DISCLOSURE

The present disclosure relates to an injection device, e.g. for manual or for spring driven injection, having an audible signal or a tactile signal or both an audible and tactile signal to indicate when a dose is considered to be fully injected and the injection can be terminated.

### BACKGROUND

Most of the injection devices on the market today, which are capable of setting doses of various sizes, visually count down to zero on a display during the injection. This allows the user to follow the progress of the injection and to determine when the mechanical parts have reached the initial zero position. When the zero position has been reached it is recommended that the user wait for 5-6 seconds to allow the pressure, which has built up in the device during the injection, to expel the full set dose out though the needle. An indication of when the mechanical parts have reached the zero position is important, as a malfunction in the device or a clogged needle might stop the injection, and give the user the impression that the full dose has been injected. This could result in the user receiving an under dose. A visible indication of the progress of the injection, however, is not always sufficient, as many users, e.g. diabetics, have reduced eyesight and because the devices often are used in positions where the display is not visible for the user. An additional audible or tactile indication of the progress of the injection is therefore preferable.

Prior art documents having mechanisms that may, in some fashion, be considered to indicate an end of dose condition include, for example, WO9938554, disclosed in these prior art documents require a user to count a number of clicks during injection. Others produce tactile feedback prematurely because the devices fail to account for internal backpressure that builds up in the device during injection. Such devices may be more accurately described as producing an end of stroke indication when a button or other injection member is moved by the user to its mechanical limit, rather than producing an end of dose indication when the full amount of the set dose has exited the needle. Still other prior art devices may be of an undesirable complexity or provide a false indication if the injection is interrupted or may actually produce a movement of a plunger drive member when the end of dose is indicated.

Based on the foregoing, there is still room for improvement in the area of end of dose feedback mechanisms.

### SUMMARY

An apparatus, system, or method may comprise one or more of the features recited in the appended claims and/or the following features which, alone or in any combination, may comprise patentable subject matter:

According to an aspect of the present disclosure, an injection device for injecting a medicament includes a housing, a dose setting member movable relative to the housing for setting a dose to be injected, and a signal part. The signal part rotates about an axis relative to a surface within the injection device from a first rotational position to a second rotational position to increase loading on a spring when a dose is set due to rotation of the dose setting member relative to the housing. An internal pressure builds up in the injection device during injection which results in the signal part being frictionally captured in the second rotational position between first and second internal parts of the injection device. After the internal pressure dissipates by a sufficient amount during injection, the signal part rotates under the urging of the loaded spring from the second rotational position back to the first rotational position. A portion of the signal part moves into contact with the surface when the signal part reaches the first rotational position to produce tactile or audible feedback indicating that an end of dose condition has been reached.

In some embodiments, the spring comprises a torsion spring coupled to the signal part and to the first internal part. A rotational tower has a track formed on an inner surface thereof, the track having an upper portion and a widened lower end. The signal part has a segment received in the track. During rotation of the dose setting member to set the dose, the signal part moves axially relative to the rotational tower so that the segment moves from the widened lower end into the upper portion of the track and the loading of the torsion spring is increased due to relative rotation between the signal part and the first internal part as the signal part moves from the first rotational position to the second rotational position. During injection, the thread segment moves into the widened lower end of the track and then, after internal pressure dissipation by a sufficient amount, the torsion spring loading decreases to move the segment of the signal part away from one side of the widened lower end toward another side that includes the surface as the signal part rotates from the second rotational position toward the first rotational position. In some embodiments, the portion of the signal part that moves into contact with the surface to produce the tactile or audible feedback comprises an axially extending edge of the signal part. The surface contacted by the axially extending edge of the signal part may comprise an axially extending edge of the second internal part, for example.

In some embodiments according to this disclosure, the signal part includes a main body having a first tab and the spring includes a spring arm of the signal part that extends from the main body in a curvilinear cantilevered manner such that the spring arm curves about the axis. The spring arm has a distal end with a second tab. The housing has a track formed on an inner surface thereof, the track having an elongated first segment and an enlarged space at an end of the first segment. After the setting of the dose and prior to injection of the dose, the first and second tabs are situated within the elongated first segment of the track and the spring arm is flexed due to the signal part having been rotated from the first rotational position to the second rotational position. During the injection, the first and second tabs move into the enlarged space of the track, and when thereafter the internal pressure in the injection device has dissipated sufficiently, the spring arm deflects to spread the first and second tabs apart, thereby to move the signal part from the second rotational position back to the first rotational position.

According to some embodiments of this disclosure, a rotational tower is located in an interior region of the housing. The rotational tower has a track formed on an inner surface thereof, the track having an elongated first segment and an enlarged space at an end of the first segment. After the setting of the dose and prior to the injection of the dose, the first and second tabs are situated within the elongated first segment of the track and the spring arm is flexed due to the signal part having been rotated from the first rotational position to the second rotational position. During the injection, the first and second tabs move into the enlarged space of the track, and when thereafter the internal pressure in the injection device has dissipated sufficiently, the spring arm deflects to spread the first and second tabs apart, thereby to move the signal part from the second rotational position back to the first rotational position.

According to some embodiments of this disclosure, the spring includes a spring arm that is coupled to the first internal part and that extends generally axially. The signal part includes a spring arm engaging portion that engages a portion of the spring arm to increase loading on the spring arm as the signal part moves from the first rotational position to the second rotational position. A free end of the spring arm has a lug formed thereon and the spring arm engaging portion of the signal part comprises an edge defining a slot that receives the lug therein.

According to an aspect of the present disclosure, an end of dose notification mechanism for an injection device used for injecting a medicament is provided. The end of dose notification mechanism includes a rotational tower that is generally tubular and that has a track formed on an inner surface thereof, the track having an elongated first segment and an enlarged space at an end of the first segment. The mechanism also includes a dose setting member movable relative to the rotational tower to set a dose to be injected and a signal part situated in an interior region of the rotational tower and movable along an axis defined by the rotational tower. The signal part has a main body with a first tab received in the track. The signal part has a spring arm cantilevered from the main body. The spring arm extends in a curved manner about the axis defined by the rotational tower and the spring arm has a distal end with a second tab.

Movement of the dose setting member to set the dose causes the main body of the signal part to rotate about the axis from a first rotational position to a second rotational position relative to the rotational tower such that the first tab is moved toward the second tab to increase loading of the spring arm. After setting of the dose and prior to injection of the dose, the first and second tabs are situated within the elongated first segment of the track of the rotational tower and, during injection, the first and second tabs move into the enlarged space. An internal pressure builds up in the injection device during injection which results in the signal part being captured in the second rotational position between first and second internal parts of the injection device such that the first and second tabs are prevented from spreading apart within the enlarged space. Then, in response to the internal pressure dissipating by a sufficient amount, the spring arm deflects to spread the first and second tabs apart, thereby to rotate the main body of the signal part from the second rotational position back to the first rotational position to click the first tab against a surface of the rotational tower within the enlarged space of the track to signal the end of dose condition being reached.

In some embodiments, the inner surface of the rotational tower is generally cylindrical and the elongated first segment forms a helical track along the inner surface. In some embodiments, the elongated first segment extends less than one revolution about the axis of the rotational tower such as extending less than 180° about the axis of the rotational tower. In other embodiments, the inner elongated first segment extends along the inner surface of the rotational tower in substantially parallel relation with the axis of the rotational tower. Alternatively or additionally, the rotational tower serves as an outer housing of the injection device.

According to some embodiments of the present disclosure, the main body of the signal part is substantially cylindrical and the signal part includes an annular flange extending radially inwardly from a top of the main body. The annular flange is clamped between first and second internal parts of the injection device due to the internal pressure. Dissipation of the internal pressure results in the annular flange being unclamped from the first and second internal parts and permits the main body of the signal part to rotate about the axis of the rotational tower in response to deflection of the spring arm.

According to another aspect of the present disclosure, an end of dose mechanism is provided for use with an injection device having at least two members that experience axial force when the injection device is operated to force medication from a cartridge of the injection device. The end of dose mechanism includes a spring and a signal part that rotationally moves from a first position to a second position during dose setting to increase loading of the spring. A portion of the signal part is frictionally captured in the second position between surfaces of the at least two members due to internal pressure that builds up in the cartridge during injection. After the internal pressure dissipates by a sufficient amount, the at least two members have released the portion of the signal part thereby to permit the signal part to rotate, under the urging of the spring, from the second position back to the first position. The signal part has a first surface that contacts a second surface of the injection device to provide tactile or audible feedback indicating that an end of dose condition has been achieved.

In some embodiments, the signal part includes a tab and the tab provides the first surface. In other embodiments, the signal part includes a notch that defines an axially extending edge and the axially extending edge provides the first surface. In some embodiments, the signal part includes a main body and the spring comprises a spring arm that extends from the main body in a cantilevered manner The spring arm is curved about the axis. In some embodiments, the spring arm and the main body are integrally formed. In other embodiments, the spring comprises a torsion spring having a first end coupled to the signal part and having a second end coupled to one member of the at least two members.

According to the present disclosure, therefore, a mechanism for an injection device gives the user feedback in terms of an audible signal or a tactile signal or both when the device has delivered the full amount of a set dose. The acoustic or tactile signal indicates that the full dose has been injected and the user is able to pull out the needle to terminate the injection. It is contemplated that the mechanism only gives the signal at the end of dose condition being reached. Thus, it will be appreciated that the end of dose condition (e.g., full amount of dose has been injected) is reached a period of time after the end of stroke condition (e.g., the point at which a button or similar such structure has been pressed or otherwise moved by a user to its mechanical limit to bring about the result of injecting a dose) has been reached. Thus, the phrase “during injection” in the present disclosure and in the claims is intended to cover the entirety of the time period that medication is delivered from the injection device which includes time periods before the end of stroke condition and time periods after the end of stroke condition, up to and including the end of dose condition.

According to a further aspect of this disclosure, an end of dose mechanism that is provided for use with an injection device includes a first part having a generally cylindrical portion with a window formed therethough. The first part has a spring arm formed integrally with the cylindrical portion and extending generally axially into the window. The first part has at least one protrusion that projects radially from the generally cylindrical portion. The end of dose mechanism has a signal part coupled to the first part for rotation between a first rotational position and a second rotational position. The signal part has a spring arm engaging portion and the signal part also has at least one space that receives the at least one protrusion therein.

During use of the injection device to set a dose, the signal part rotates from the first rotational position to the second rotational position and the spring arm engaging portion acts upon the spring arm to move the spring arm within the window to increase loading of the spring arm. During use of the injection device to inject a medication, an internal pressure builds up in the injection device during injection which results in the signal part being maintained in the second rotational position such that the spring arm is prevented from moving to decrease its loading. In response to the internal pressure dissipating by a sufficient amount, the spring arm deflects and acts upon the spring arm engaging portion to rotate the signal part from the second rotational position back to the first rotational position to click an edge of the signal part against a surface of the protrusion received in the at least one space of the signal part.

In some embodiments, a free end of the spring arm has a lug formed thereon and the spring arm engaging portion of the signal part comprises an edge defining a slot that receives the lug therein. A thread segment is formed on the at least one protrusion. At least one thread segment is formed on the signal part. The signal part has at least one arm adjacent the at least one space and the at least one thread is formed on the at least one arm.

In some embodiments, the at least one protrusion is situated adjacent a first end of the first part. Fhe first part has at least one snap finger that extends generally axially at a second end of the first part and the at least one snap finger has a ramped flange formed thereon. The end of dose mechanism further includes a second part having a window that receives the ramped flange therein to connect the first and second parts together. The signal part is trapped between the at least one surface of the first part and an annular edge of the second part.

Additional features, which alone or in combination with any other feature(s), such as those listed above and those listed in the claims, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures, in which:

FIG. 1 is a schematic vertical sectional view of a first embodiment of the mechanism according to the present disclosure, showing a device in which a dose has been set;

FIG. 2 is a schematic vertical sectional view of the first embodiment of the mechanism according to the present disclosure, similar to FIG. 1, showing the device in a state in which a button at the top of the device has been pressed downwardly to initiate injection of the dose;

FIG. 3 is a schematic perspective view of a signal part according to a first embodiment of the present disclosure;

FIG. 4 is a schematic vertical sectional view of a rotational tower of a first embodiment according to the present disclosure;

FIG. 5 is a schematic vertical sectional view of a second embodiment of the mechanism according to the present disclosure, showing a device in which a dose has been set;

FIG. 6 is a schematic vertical sectional view of the second embodiment of the mechanism according to the present disclosure, showing the device in a state in which a button at the top of the device has been pressed downwardly to initiate injection of the dose;

FIG. 7 is a schematic perspective view of the signal part according to the second embodiment of the present disclosure;

FIG. 8 is a schematic vertical sectional view of a housing of the second embodiment according to the present disclosure;

FIG. 9 is a schematic vertical sectional view of a third embodiment of the mechanism according to the present disclosure, showing a device in which a set dose has been injected;

FIG. 10 is an exploded view of the third embodiment according to the present disclosure;

FIG. 11 is a perspective view of a gearing mechanism of the third embodiment according to the present disclosure after the completion of an injection and before rotation of the signal part;

FIG. 12 is a perspective view of the gearing mechanism of the third embodiment according to the invention after the completion of an injection and after rotation of the signal part;

FIG. 13 is a schematic partial sectional view of the rotational tower of FIG. 4 showing first and second tabs of the signal part of FIG. 3 being situated in an elongated segment of a track formed in a cylindrical inner surface of the rotational tower and a downward arrow indicating a direction of movement of the first and second tabs within the track when a dose is injected;

FIG. 14 is a schematic partial sectional view, similar to FIG. 13, showing the first and second tabs being situated in an enlarged space at a lower end of the track of the rotational tower when a dose is initially injected, the first and second tabs being held in place due to a clamping force that acts upon another portion of the signal part and that prevents rotation of the signal part relative to the rotational tower;

FIG. 15 is a schematic partial sectional view, similar to FIG. 14, showing the first tab separating from the second tab in the direction of the arrow to snap against a surface of the rotational tower when the clamping force dissipates by a sufficient amount;

FIG. 16 is a schematic partial sectional view, similar to FIG. 15, showing the first and second tabs moving back toward the elongated segment of the track in the direction of the upwardly oriented arrow with the first tab being moved in the direction of the horizontally oriented arrow due to contact with a ramped surface of the enlarged space of the track;

FIG. 17 is a schematic partial sectional view, similar to FIG. 16, showing the first and second tabs moved back into the elongated segment of the track during further upward movement in the direction indicated by the arrow;

FIG. 18 is a schematic partial sectional view of the housing of FIG. 8 showing first and second tabs of the signal part of FIG. 7 being situated in an elongated segment of a track formed in a cylindrical inner surface of the rotational tower and a downward arrow indicating a direction of movement of the first and second tabs within the track when a dose is injected;

FIG. 19 is a schematic partial sectional view, similar to FIG. 18, showing the first and second tabs being situated in an enlarged space at a lower end of the track of the housing when a dose is initially injected, the first and second tabs being held in place due to a clamping force that acts upon another portion of the signal part and that prevents rotation of the signal part relative to the housing;

FIG. 20 is a schematic partial sectional view, similar to FIG. 19, showing the first tab separating from the second tab in the direction of the arrow to snap against a surface of the housing when the clamping force dissipates by a sufficient amount;

FIG. 21 is a schematic partial sectional view, similar to FIG. 20, showing the first and second tabs moving back toward the elongated segment of the track in the direction of the upwardly oriented arrow with the first tab being moved in the direction of the horizontally oriented arrow due to contact with a ramped surface of the enlarged space of the track;

FIG. 22 is a schematic partial sectional view, similar to FIG. 21, showing the first and second tabs moved back into the elongated segment of the track during further upward movement in the direction indicated by the arrow;

FIG. 23A is an exploded perspective view of a fourth embodiment of an end of dose mechanism according to the present disclosure showing a connector lock at the top of the page, an end of dose click tube or signal part beneath the connector lock, and a tubular connector beneath the signal part, the connector having a generally axially extending spring arm situated in a window formed in the connector tube, and the spring arm having a knob or lug at its upper end;

FIG. 23B is an exploded perspective view of the four embodiment, similar to FIG. 23A, but at a different angle so that radially inwardly extending tabs at the upper end of the signal part are viewable; and

FIG. 24 is a perspective view of the assembled fourth embodiment showing the lug at the end of the spring arm being situated in an axially extending slot of the click tube and showing the connector having pads with helical thread portions occupying a portion of respective notches provided at the bottom of the click tube.

### DETAILED DESCRIPTION

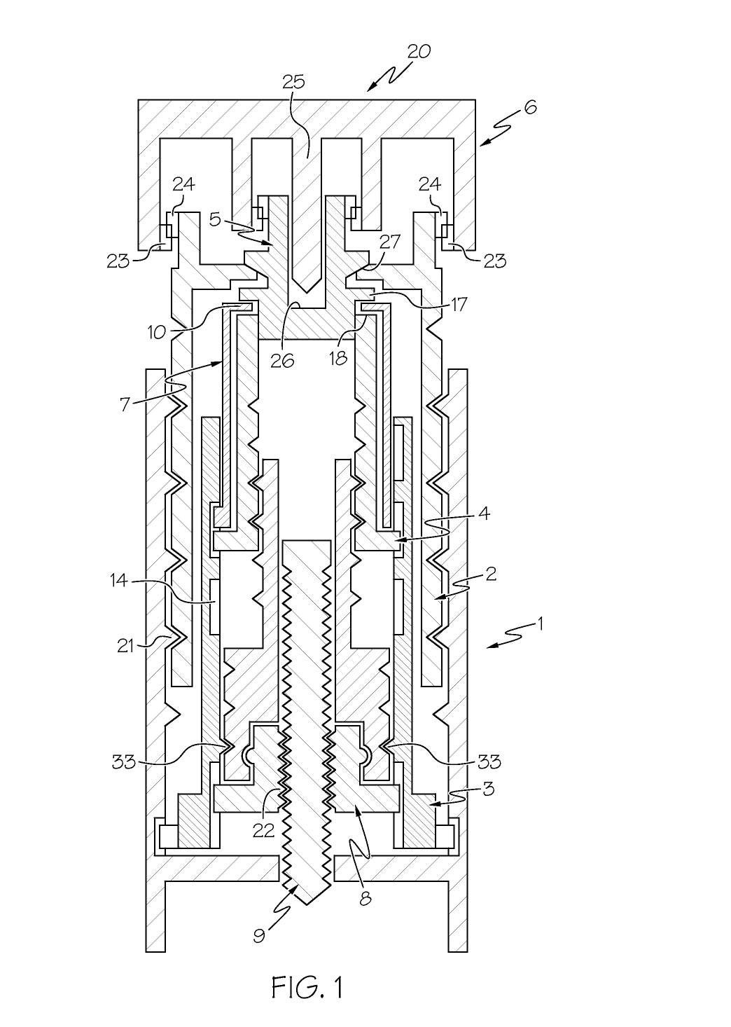

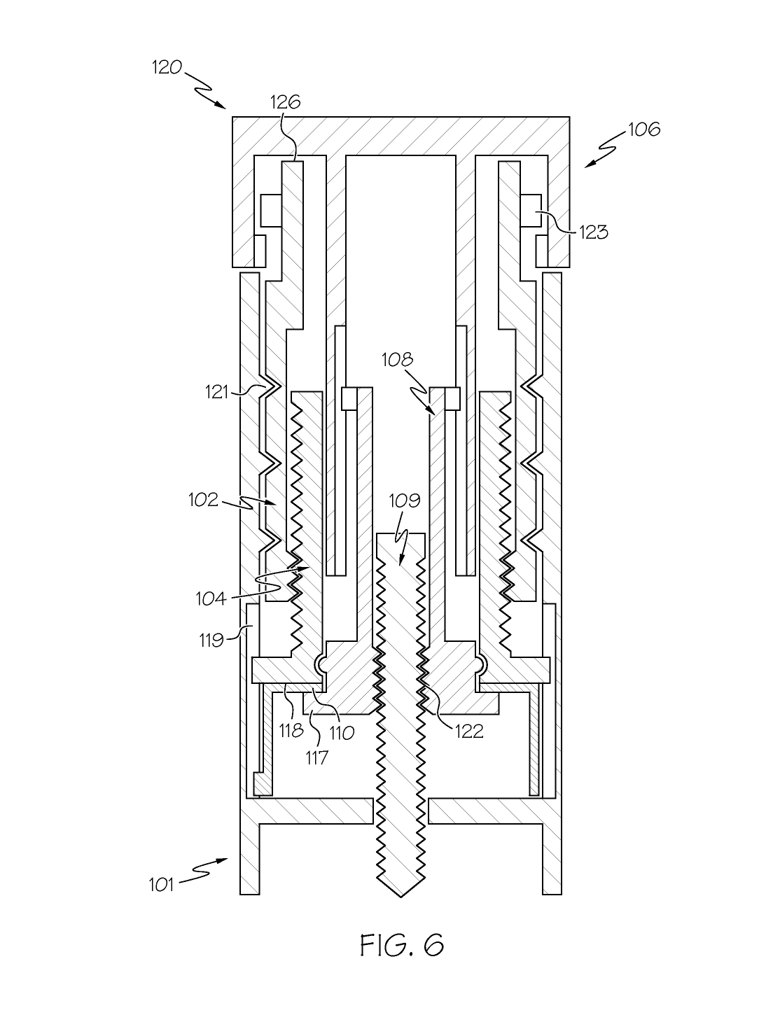

In the following the term main axis defines the common axis for the mainly tube shaped parts and for the entire injection device. Primarily, only the parts related to understanding the function of the signal feature of the end of dose notification mechanism is included in the description, however, the drawings may show other parts which could be part of an injection device comprising the feature. The disclosed injection devices of FIGS. 1-4 and FIGS. 9-12 are similar to those disclosed in WO 2012/037938 A1 which is hereby incorporated by reference herein, while the injection device of FIGS. 6-8 is similar to those disclosed in WO 2005/018721, which is hereby incorporated by reference herein.

The terms “up” and “down” and “upper” and “lower” and “upward” and “downward” refer to the drawings and not to a situation of use.

In all embodiments the described screw is abutting a plunger in a medicine filled cartridge and downward movement of the screw moves the plunger in the cartridge and medicine is pressed out through a needle. The plunger, cartridge and needle are not shown in the drawings but are well known in the art.

The dose selector and the push-button may be two separate parts or may be one part having two functions.

FIG. 1 shows a first embodiment of an injection device according to this disclosure with the mechanism arranged on the geared side of a device and in which a dose to be injected has been set. A dial 2 engages a housing 1 via a first thread connection 21 and a non-rotational screw 9 engages a dosage nut 8 via a second thread connection 22. The thread pitch of the first thread connection 21 is bigger than the thread pitch of the second thread connection 22 and the axial displacement of the dial 2 per set unit is bigger than the axial displacement of the dosage nut 8 per unit, for example, with a ratio between the movements at 3:1 in some embodiments but with other ratios greater than or less than 3:1 being within the scope of the present disclosure. When a dose is set, the dosage nut 8 rotates and when a set dose is injected the dosage nut 8 does not rotate, whereby it will simply press down the screw 9 the non-geared distance, while the dial 2 will be rotated down moving the geared distance.

As can be seen in FIG. 1, a primary driver 4 and a secondary driver 5 are rotationally locked together, and these two parts, together with a signal part 7, has moved the geared distance along with the dial 2 during dose setting. To set the dose, a dose setting member 6 is rotated which, in turn, rotates the dial 2 and the primary and secondary drivers 4, 5 via disengageable teeth connections 23, 24. It can also be seen that the dosage nut 8, at the same time, has moved the non-geared distance. The secondary driver 5 is capable of moving a small axial distance relative to the primary driver 4 and a flange 10 on the signal part 7 becomes locked between the upper surface 18 of the primary driver 4 and a flange 17 on the secondary driver 5 during injection of the dose and for a short time thereafter due to backpressure or internal pressure from the medication in the injection device. A spring (not shown) biases a push-button 20 of the injection device away from part 5 in a well-known manner after the user has released the pressure from the push-button 20.

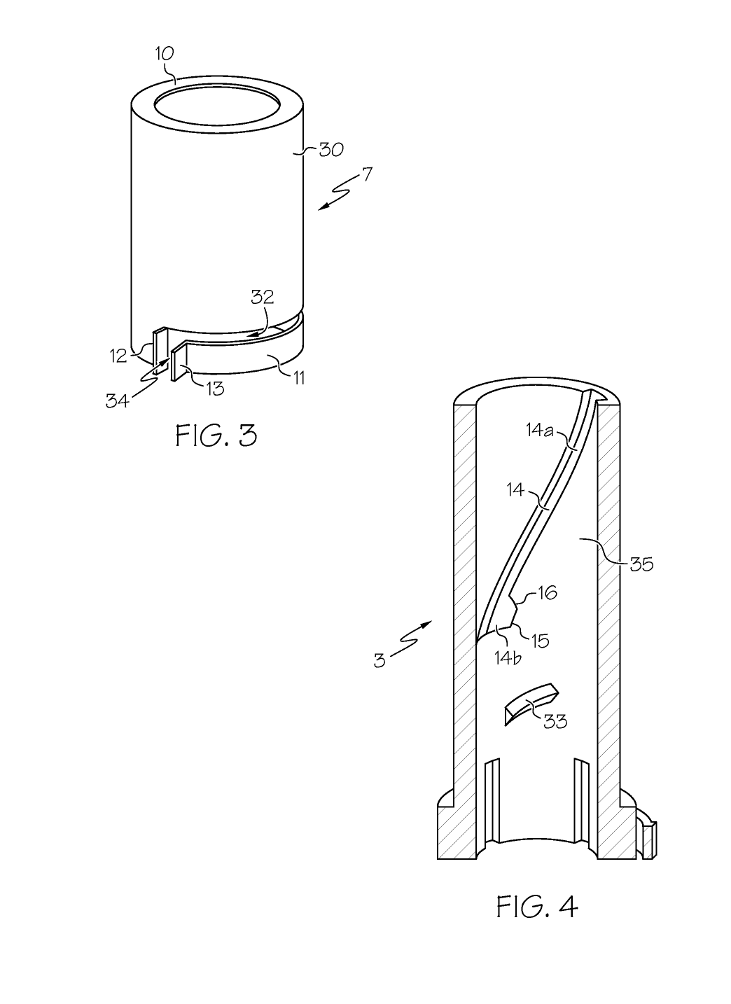

FIG. 3 shows a perspective view of the signal part 7 which is the primary component of the end of dose notification mechanism of injection device 20. The signal part 7 is made of sheet metal in some embodiments and, as can be seen in FIG. 3, the lower part comprises a spring arm 11 with a bend in the free end forming a spring key or tab 13. Spring arm 11 extends in a curved, cantilevered manner from a main body 30 of signal part 7. Thus, an end of spring arm 11 that is opposite of the end having tab 13, is integral with a lower end region of main body 30. That is, spring arm 11 and main body 30 are formed integrally so as to be a unitary piece. On the main body of the signal part 7, a body key or tab 12 is provided. The main body 30 of part 7 is substantially cylindrical in shape. Signal part 7 is formed to include a circumferentially extending slot 32 that is located above spring arm 11 and beneath main body 30. Signal part 7 also has an axially extending slot 34 located between tabs 12, 13. It is the provision of slots 32, 34 in signal part 7 that gives spring arm 11 its flexibility relative to the main body 30. It should be apparent in FIG. 3 that the curvature of spring arm 11 and of main body 30, in general, is centered on the main axis of the injection device 20. The annular flange 10 of signal part 7 extends radially inwardly from the upper end of main body 30 toward the main axis of the injection device 20.

Referring again to FIG. 1, the primary driver 4 engages a rotational tower 3 in a third thread connection 14 with an even higher pitch than the first thread connection 21. The rotational tower 3 is sometimes referred to herein as the “click tower.” The rotational or click tower 3 is another component of the end of dose notification mechanism (sometimes referred to as simply an “end of dose mechanism”). The rotational tower 3, the primary driver 4, and the dosage nut 8 are arranged in such a way that when the primary driver 4 is moved the geared distance, it will move the dosage nut 8 the non-geared distance by means of one or more intermediate parts. FIG. 4 shows a sectional view of the rotational tower 3. The internal thread or helical track forming the third thread connection 14 together with the primary driver 4 is visible, and it can clearly be seen how the thread or track 14 widens in the lower end via an inclined transition 16. Thus, the track 14 includes an elongated first segment or narrow area 14a and an enlarged space 14b at the lower end of the first segment 14a. The term “thread” and “track” as used herein, including in the claims, is intended to cover male threads and male tracks, respectively, as well as female threads and female tracks, respectively.

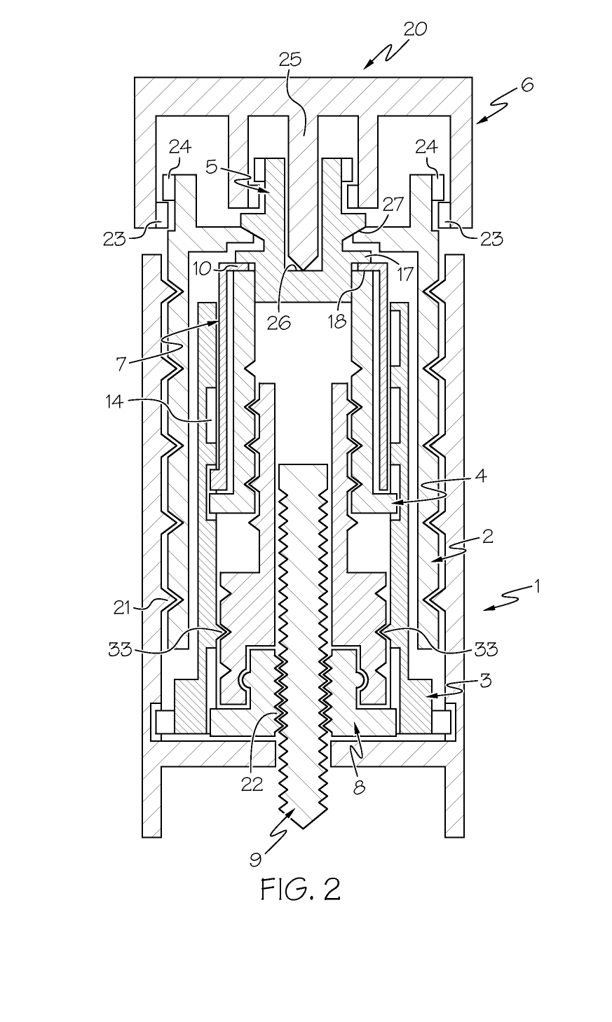

In the embodiment of FIG. 4, the elongated first segment 14a of thread 14 forms a helical track along an inner surface 35 of the click tower 3. The helical track of the elongated first segment 14a extends less than 180° about the main axis of the click tower 3. In the illustrative example, track 14 extends about 120° about the main axis. In other embodiments, a track similar to track 14 extends more than 180° or less than 120° about the main axis of the associated click tower, as desired. The rotational tower 3 also has a helical segment 33 that protrudes inwardly from the inner surface 35. Helical segment 33 is situated generally between the enlarged space 14b and a lower end of the click tower 3 in the illustrative example. As shown in FIGS. 1 and 2, there are two helical segments 33 that protrude from the inner surface 35 of the rotational tower 4 but only one of these can be seen in FIG. 4. The helical segments 33 form a threaded connection with one of the intermediate parts of the injection device as shown in FIGS. 1 and 2.

The spring key 13 and the body key 12 engage the internal thread 14 of the rotational tower 3 and, when a dose is set, the signal part 7 rotates from a first rotational position to a second rotational position relative to the rotational tower 3. In the illustrative example, the signal part 7 also moves axially during dose setting. As the signal part 7 rotates from the first rotational position to the second rotational position, the keys 12, 13 move from the wide area 14b of the thread 14 to the narrow area 14a of the thread which, in turn, tenses or increases the loading of the spring arm 11 by deflecting it relative to main body 30. As signal part 7 moves further axially during dose setting, the helical shape of track 14a causes signal part 7 to undergo further rotation about the axis of injection device 20 as tabs 12, 13 move upwardly within the track 14a. However, during this further axial movement of signal part 7, spring arm 11 continues to be loaded or tensed by substantially the same amount because the distance between tabs 12, 13 remains substantially constant as they move upwardly within track 14a. During injection, signal part 7 moves axially downwardly such that tabs 12, 13 move downwardly in track 14a with a resultant rotation of signal part 7 in an opposite direction to that which occurred during dose setting.

In FIG. 2, injection of the dose has been initiated. To inject the set dose, the push-button 20 is pushed downwardly toward housing 1. This downward movement of button 20 disengages the teeth connections 23, 24 between the dose setting member 6 and the dial 2 and between the dose setting member 6 and the secondary driver 5. Furthermore, a shaft 25 of the combined push-button 20 and dose setting member 6 is pushed downwardly to engage a surface 26 of the secondary driver 5 such that further downward pushing of the dose setting member 6 pushes down the secondary driver 5 as well. The downward movement of the secondary driver 5, due to the continued pushing on the dose setting member 6, also pushes down the dial 2 via the sliding surface connection 27 and the primary driver 4 through the flange 10 of the signal part 7. Thereby, the force applied by the user to inject a dose is transmitted through the flange 10 and a frictional torque is applied to the signal part 7.

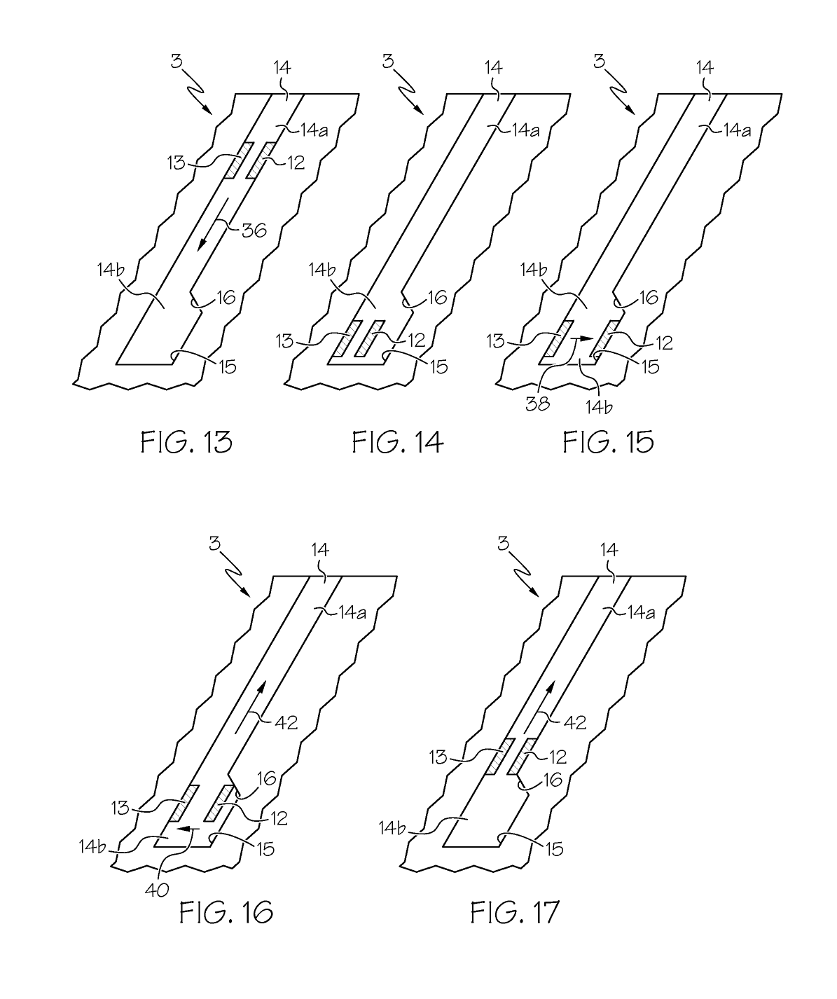

When all of the parts that move the geared distance (e.g., dose setting member 6, dial 2, primary and secondary drivers 4, 5, and signal part 7) have been pushed all the way to the zero position, the movement is stopped by a rotational stop between the dial 2 and the housing 1, but because of the internal pressure which has built up in the cartridge during the injection due to the hydraulic resistance in the needle and, in some embodiments, because of the compression of the spring (not shown) between button 20 and driver 5, the flange 10 of the signal part 7 and the primary and secondary drivers 4, 5 are still pressed together and a frictional torque is still applied to the signal part 7 with flange 10 being frictionally captured between internal parts 4, 5 of device 20. At this point, the keys 12, 13 of the signal part 7 have moved downwardly in the direction of arrow 36, shown in FIG. 13, from a starting position within narrow area 14a of thread 14, into the wide area 14b of the internal thread 14 of the rotational tower 3, as shown in FIG. 14, and the flexed spring arm 11 continues to apply a torque to the signal part 7. FIG. 14 corresponds to an end of stroke condition of the injection device. However, the frictional torque imparted on the signal part 7 at flange 10 is bigger than the torque applied by the spring arm 11, and the signal part 7 does not rotate until the pressure in the cartridge has dissipated or been reduced to a level having the frictional torque on flange 10 lower than the torque applied by the flexed spring arm 11. When that situation occurs, main body 30 and flange 10 of the signal part 7 will rotate rapidly, as indicated by arrow 38 in FIG. 15, from the second rotational position back to the first rotational position and the body key 12 of the main body 30 of the signal part 7 will move into abutment with the surface 15 of the wide area 14b of the internal thread 14 of the rotational tower 3 resulting in both a tactile and an audible signal (e.g., a click), which informs the user that the injection has been fulfilled and the needle can be retracted from the skin. Thus, FIG. 15 corresponds to the end of dose condition of the injection device.

When a new dose is set, signal part 7 moves upwardly within the injection device and the tab 12 rides along the inclined surface 16 so that keys 12, 13 are squeezed together again, as indicated by arrow 40 shown in FIG. 16, and then tabs 12, 13 move upwardly into narrow area 14a of thread 14 as indicated by arrow 42 shown in FIGS. 16 and 17. As tabs 12, 13 are squeezed together, flexible arm 11 is flexed or tensed once again. At that point, the signal part 7 is loaded and ready to give a signal at the next end of dose situation.

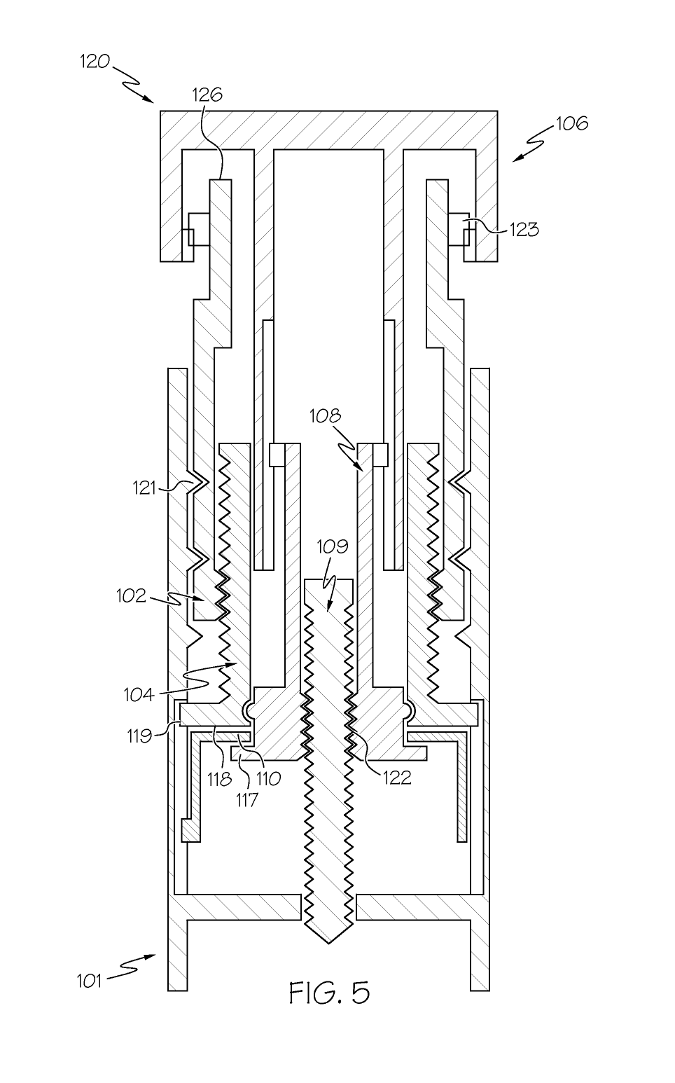

In a second embodiment shown in FIGS. 5 and 6, the mechanism that indicates an end of dose condition is arranged on the non-geared side of an injection device. FIG. 5 shows such a configuration of an injection device, in which a dose to be injected has been set. A dial 102 engages a housing 101 in a first thread connection 121 and a dosing nut 108 engages a non-rotational screw 109 in a second thread connection 122. The thread pitch of the first thread connection 121 is bigger than the thread pitch of the second thread connection 122 and the axial displacement of the dial 102 per set unit is bigger than the axial displacement of the dosage nut 108 per unit e.g. with a ratio between the movements at 3:1 in some embodiments but with other ratios greater than or less than 3:1 being within the scope of the present disclosure. When a dose is set, the dosage nut 108 is forced to rotate. During an injection, the dial 102 will rotate downwardly moving the geared distance. An intermediate part 104, which moves the non-geared distance together with the dosage nut 108 during dose setting and during injection, is arranged between the dial 102 and the dosage nut 108 to transfer force therebetween during injection. The function of the intermediate part 104 is to transmit the force applied by the user and to gear down the linear displacement. During an injection, the dosage nut will be moved down by part 104 to press down the screw 109 the non-geared distance.

To set the dose, a dose setting member 106, which is rotationally locked to the dosage nut 108, is rotated and, due to a teeth connection 123 between the parts 102, 106, this will rotate the dial 102 as well and cause it to elevate the geared distance out of the housing 101 together with the dose setting member 106. The intermediate part 104 and the dosage nut 108 together with a signal part 107 will move upwardly by a non-geared distance. The dosage nut 108 is capable of moving a small axial distance relative to the intermediate part 104 and a flange 110 on the signal part 107 becomes locked or frictionally captured between a lower surface 118 of the intermediate part 104 and a flange 117 of the dosage nut 108 during injection of the dose and for a short time thereafter due to the internal pressure from the medication in the injection device. A spring (not shown) spring biases a push-button 120 of the injection device away from part 102 in a well-known manner

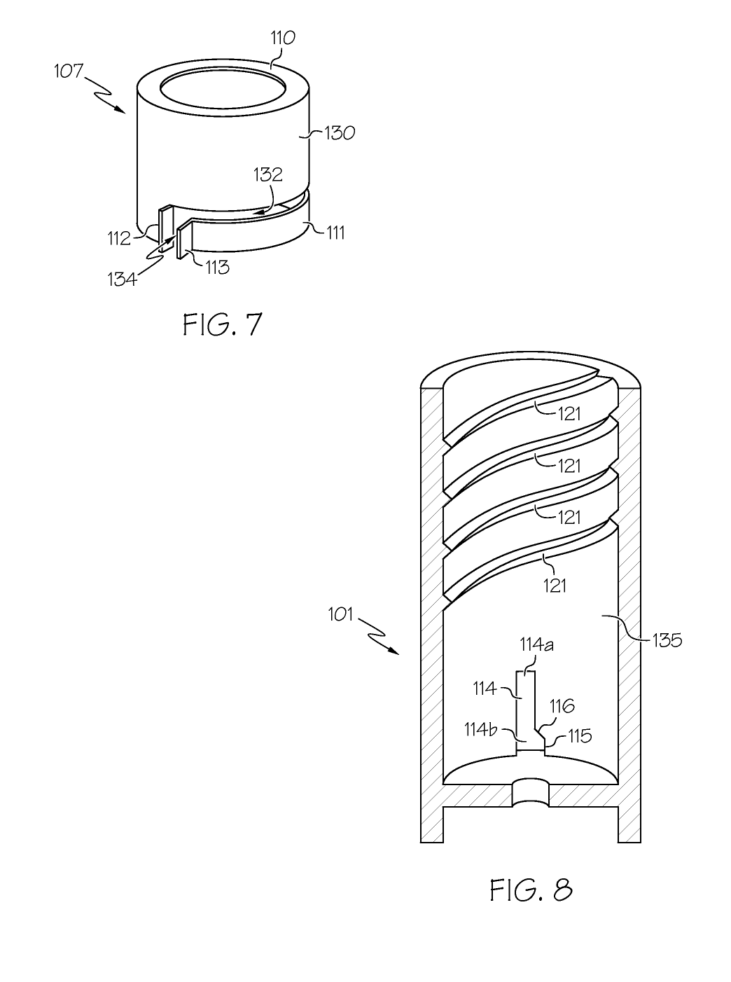

FIG. 7 shows a perspective view of the signal part 107 which is similar to signal part 7 of the first embodiment. Signal part 107 is the primary component of the end of dose notification mechanism of injection device 120. The signal part 107 is made of sheet metal in some embodiments and, as can be seen in FIG. 7, the lower part comprises a spring arm 111 with a bend in the free end forming a spring key or tab 113. Spring arm 111 extends in a curved, cantilevered manner from a main body 130 of signal part 107. Thus, an end of spring arm 111 that is opposite of the end having tab 113, is integral with a lower end region of main body 130. That is, spring arm 111 and main body 130 are formed integrally so as to be a unitary piece. On the main body 130 of the signal part 107, a body key or tab 112 is provided. The main body 130 of part 107 is substantially cylindrical in shape. Signal part 107 is formed to include a circumferentially extending slot 132 that is located above spring arm 111 and beneath main body 130. Signal part 107 also has an axially extending slot 134 located between tabs 112, 113. It is the provision of slots 132, 134 in signal part 107 that gives spring arm 111 its flexibility relative to the main body 130. The curvature of spring arm 111 and of main body 130, in general, is centered on the main axis of the injection device 120. The annular flange 110 of signal part 107 extends radially inwardly from the upper end of main body 130 toward the maim axis of the injection device 120.

FIG. 8 shows a sectional view of the rotational housing 101 which, in this embodiment, also forms the exterior housing of the device. An inner surface 135 of the housing 101 is formed to include a track 114 having an elongated first segment 114a and an enlarged or widened space 114b at the lower end of the first segment 114a. Unlike the helical path of portion 14a of track 14 of the first embodiment, the elongated portion 114a of track 114 is straight and extends axially relative to housing 101. The track 114 widens in the lower end via an inclined transition 116. The spring key 113 and the body key 112 engage the internal track 114 of the housing 101, and when a dose is set, the signal part 107 rotates from a first rotational position to a second rotational position. In the illustrative example, the signal part 107 also moves axially during dose setting. As the signal part 107 rotates from the first rotational position to the second rotational position, the keys 112, 113 move from the wide area 114b of the track 114 to the narrow area 114a of the track 114 which, in turn, tenses or increases the loading of the spring arm 111 by deflecting it relative to the main body 130. As signal part 107 moves further axially during does setting, signal part remains in the second rotational position because track 114a is straight and extends axially. During injection, signal part 107 moves axially downwardly such that tabs 112, 113 move downwardly in track 114a.

In FIG. 6, injection of the dose has been initiated. To inject the set dose the push-button 120 is pushed downwardly toward housing 101. This downward movement disengages the teeth connections 123 between the dose setting member 106 and the dial 102 and the push-button 120 engages the dial 102 at a sliding surface 126, such that further downward pushing on the push-button 120 also pushes down the dial 102 as well. The downward movement of the dial 102 due to the continued pushing on the push-button 120 also pushes down the intermediate part 104 and the dosage nut 108 through the flange 110 on the signal part 107. Thereby, the force applied by the user to inject a dose is transmitted through the flange 110 and a frictional torque is applied to the signal part 107.

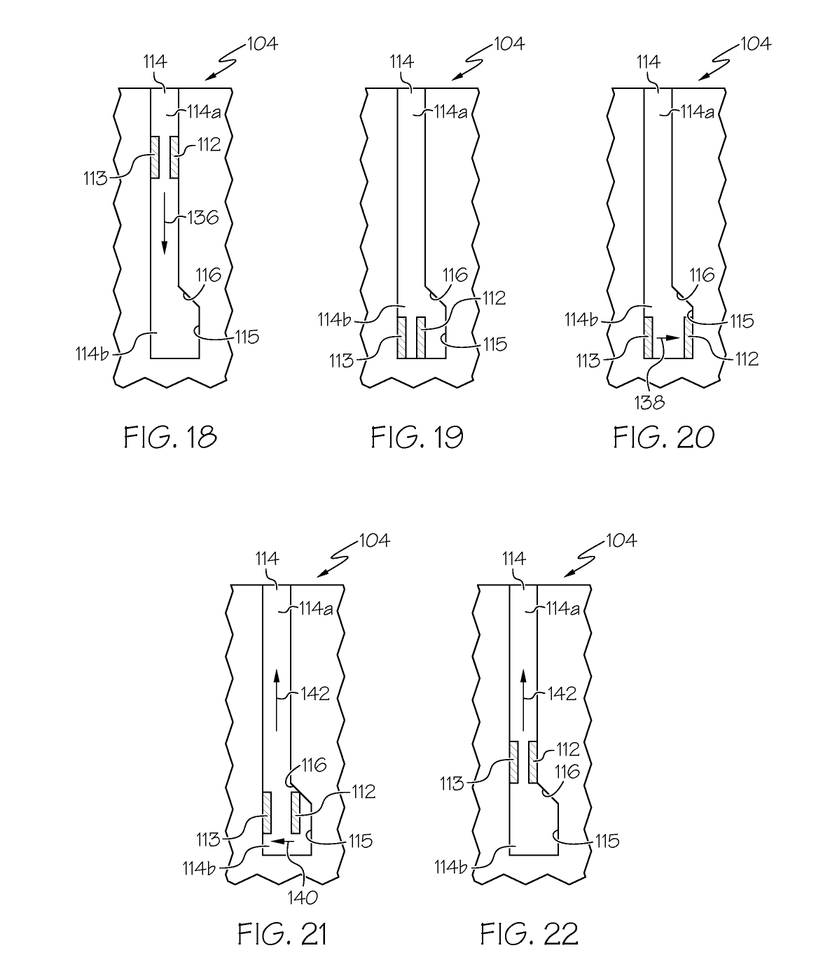

When the dose setting member 106 and the dial 102 have been pushed all the way to the zero position, the movement is stopped by a rotational stop between the dial 102 and the housing 101, but because of the internal pressure which has built up in the cartridge during the injection due to the hydraulic resistance in the needle, the flange 110 of the signal part 107, the intermediate part 104, and the flange 117 of the dosage nut 108 are still pressed together and a frictional torque is still applied to the signal part 107 with flange 110 being frictionally captured between internal parts 104, 108 of device 120. At this point, the keys 112, 113 of the signal part 107 have moved downwardly in the direction of arrow 136, shown in FIG. 18, from a starting position within narrow area 114a of track 114, into the wide area 114b of the internal track 114 of the housing 101, as shown in FIG. 19, and the flexed spring arm 111 continues to apply a torque to the signal part 107. FIG. 19 corresponds to an end of stroke condition of the injection device. However, the frictional torque imparted on the signal part 107 at flange 110 is bigger than the torque applied by the spring arm 111, and the signal part 107 does not rotate until the pressure in the cartridge has dissipated or been reduced to a level having the frictional torque on flange 110 lower than the torque applied by the flexed spring arm 111. When that situation occurs, main body 130 and flange 110 of the signal part 107 will rotate rapidly, as indicated by arrow 138 in FIG. 20, from the second rotational position back to the first rotational position and the body key 112 of the main body 130 of the signal part 107 will move into abutment with the surface 115 of the wide area 114b of the internal track 114 of the housing 101 resulting in both a tactile and an audible signal (e.g., a click), which informs the user that the injection has been fulfilled and the needle can be retracted from the skin. Thus, FIG. 20 corresponds to the end of dose condition of the injection device.

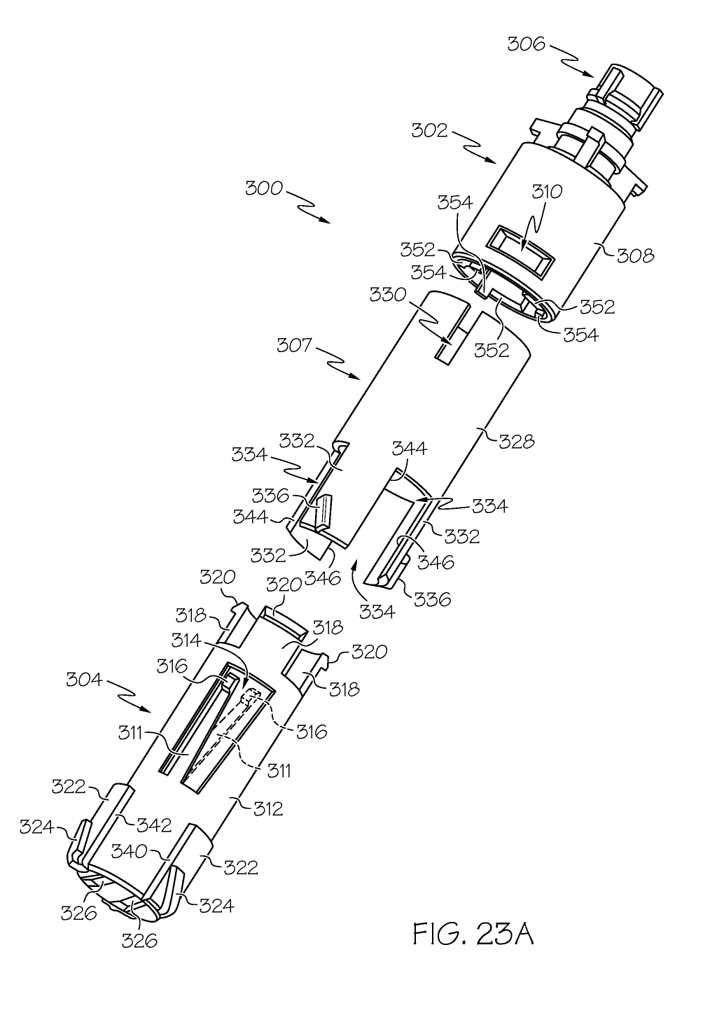

When a new dose is set, signal part 107 moves upwardly within the injection device and tab 112 rides along the inclined surface 116 so that keys 112, 113 are squeezed together again, as indicated by arrow 140 shown in FIG. 21, and then keys 112, 113 move upwardly into narrow area 114a of track 114 as indicated by arrow 142 shown in FIGS. 21 and 22. As tabs or keys 112, 113 are squeezed together, flexible arm 111 is flexed or tensed once again. At that point, the signal part 107 is loaded and ready to give a signal at the next end of dose situation. As the feature in this embodiment is arranged at the non-geared side of the device, a user must set a higher dose than is the case in the first embodiment to fully load the mechanism and prepare it for the next signal. That is, it takes more rotation of dose setting member 106 to get tabs 112, 113 to move upwardly from the wide area 114b of track 114 into the narrow segment 114a than it takes rotation of dose setting member 6 to get tabs 12, 13 to move from the wide area 14b of track 14 upwardly into the narrow segment 14a.

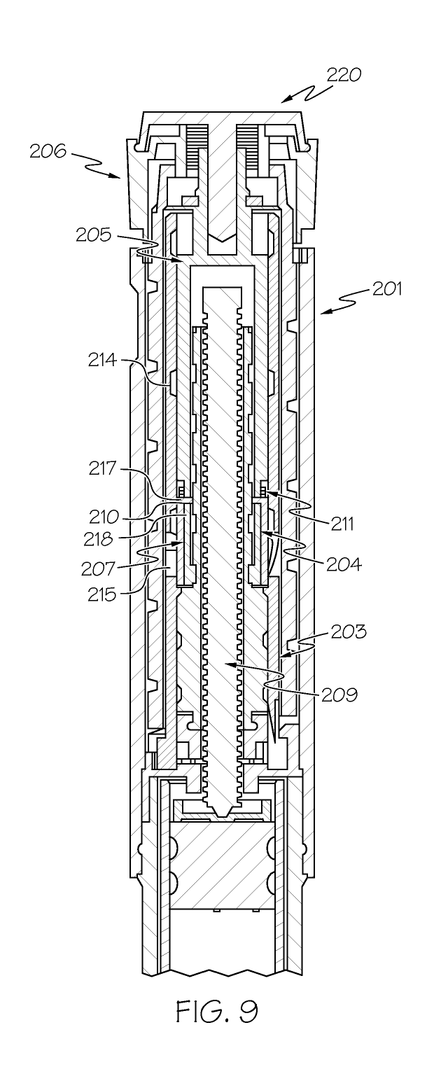

FIGS. 9-12 show a third embodiment of an end of does signaling or notification mechanism situated on the geared side of an injection device 220, which is comparable to the first embodiment regarding function, but with a torque spring 211 being a separate part that is not integrated with a signal part 207. The torque spring 211 is fixed to the signal part 207 at one end and to a secondary driver 205 at the other end.

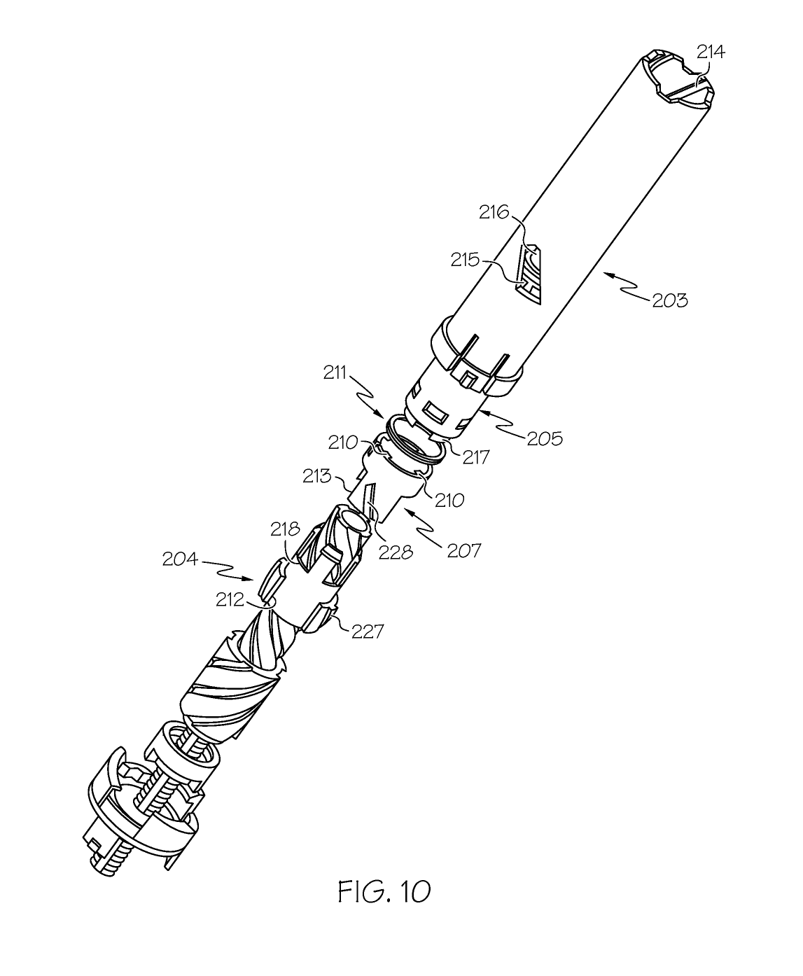

As shown in FIG. 10, a non-elevating rotational tower 203 comprises a four-start thread 214 with a high pitch and with two of the starts widening up 215 in the lower end via inclined transitions 216. To ease the molding process the widened area 215 are made as cut outs in the rotational tower 203. The signal part 207 and a primary driver 204 follow each other axially but can rotate a limited angle relative to each other. The signal part 207 has two thread segments 228 that engage two of the four-starts of the thread 214 on the rotational tower 203, which is widened up in the one end, and a primary driver 204 has two thread segments 227, which engage the two remaining starts. The thread segments 228 of the signal part 207 are positioned in the widened area 215 before a dose is set. At this point, the torque spring 211 is tensed or loaded less than it is when the dose is set. In other words, in the illustrative example, there is some tension in spring 211 at all times with the level of tension increasing when the dose is set. In FIG. 9, the relative positions of the parts can be seen in an interior region of housing 201. When a dose is set by rotating a dose elector 206, the signal part 207 and the primary driver 204 are both elevated relative to the rotational tower 203 and consequently, the thread segments 228 of the signal part 207 are rotated into the narrow area of the thread 214 via the inclined transitions 216, and the signal part 207 is thereby rotated an angle relative to the primary driver 204 from a first rotational position to a second rotational position against the biasing torque of the torque spring 211. This relative rotation further tenses or increases the loading of the torque spring 211.

To inject a set dose a user presses the push-button 220, whereby after an initial movement of the push-button 220, the push force is transmitted to the secondary driver 205. The signal part 207 has a number of protrusions 210 protruding toward the main axis of the device and positioned to be between the surfaces 218 on the primary driver 204 and protrusions 217 on the secondary driver 205 (see FIG. 9). As a result, the push force is transmitted from the secondary driver 205 through the protrusions 210 and to the primary driver 204. From the primary driver 204 the force is transmitted through a number of intermediate parts to the screw 209 and to the piston in the cartridge. Immediately after the push-button 220 has been pushed to the zero position or end of stroke position, the continued pressure from the user on the push-button 220 and the internal pressure or backpressure from the cartridge due to, for example, compression of the rubber piston in the cartridge and the hydraulic resistance in the needle, squeezes the protrusions 210 of the signal part 207 between drivers 204, 205 and prevents the signal part 207 from rotating back to the initial position (i.e., the first rotational position prior to the dose being set). Thus, at this point, protrusions 210 are frictionally captured between internal parts 204, 205 of device 220.

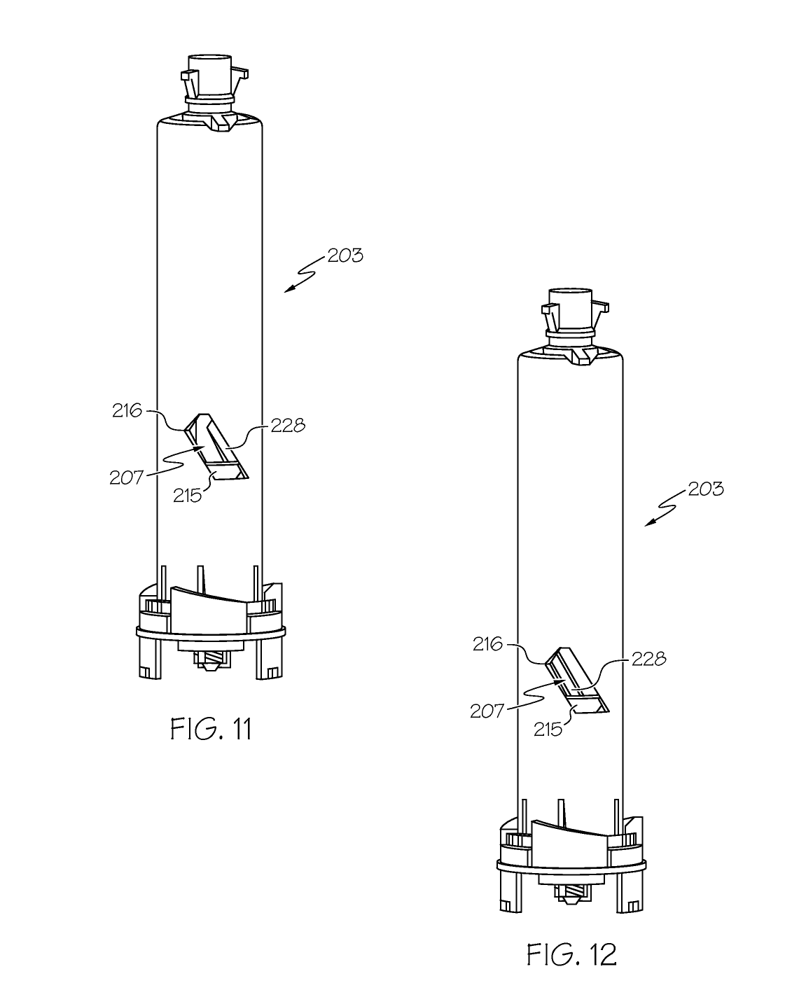

In FIG. 11, it can be seen that the segments 228 of the signal part 207 have moved down into the widened area 215 of the thread 214, but the signal part 207 is still locked against rotation. Slowly, the compressed piston will dispense the remaining dose out through the needle and the pressure on the protrusions 210 of the signal part 207 will dissipate, and when the force is low enough, it is no longer capable of holding the signal part 207 against the torque imparted on it by the torque spring 211, and the signal part 207 will start rotating. When the protrusions 210 have rotated through an angle, which is sufficiently large enough to become free of the pressure from the protrusions 217 of the secondary driver 205, the rotation of the signal part 207 will speed up during rotation from the second rotational position back to the first rotational position, and an axial surface 213 of the signal part 207 will move into abutment with an axial surface 212 of the primary driver 204 to produce an audible or tactile signal (e.g., a click). This is the signal to the user that the full dose has been injected, and that the needle can be pulled out from the skin. In FIG. 12, it can be seen that one of the thread segments 228 of the signal part 207 has rotated in to an opposite side of the respective widened area 215 as compared to the position of the segment 228 in FIG. 11. In the illustrative example, axial surface 213 serves as a portion of a boundary for a notch provided in a lower region of signal part 207.

Based on the foregoing, it should be appreciated that injection devices 20, 120, 220 each have an end of dose notification mechanism that includes respective signal parts 7, 107, 207. Each of the signal parts 7, 107, 207 rotates about an axis relative to the respective housing 1, 101, 201 from a first rotational position to a second rotational position to increase loading on the respective spring (e.g., spring arms 11, 111 and torsion spring 211) when a dose is set due to rotation of the dose setting member 6, 106, 206 relative to the respective housing 1, 101, 201. An internal pressure builds up in the injection device 20, 120, 220 during injection which results in the respective signal part 7, 107, 207 being frictionally captured in the second rotational position between first and second internal parts (e.g., 4, 5; 104, 108; and 204, 205) of the respective injection device 20, 120, 220. After the internal pressure dissipates by a sufficient amount during injection, the signal part 7, 107, 207 is released for rotation relative to the respective housing 1,101, 201 under the urging of the corresponding loaded spring 11, 111, 211 from the second rotational position back to the first rotational position. A portion (e.g. tabs 12, 112 and axial surface 213) of the respective signal part 7, 107, 207 moves into contact with an associated surface (e.g., surfaces 15, 115, 212) when the respective signal part 7, 107, 207 reaches the first rotational position to produce tactile or audible feedback indicating that an end of dose condition has been reached.

In some embodiments, rotation of the signal part relative to the exterior housing does not need to occur if the proper rotation occurs relative to one or more other internal parts of the injection device.

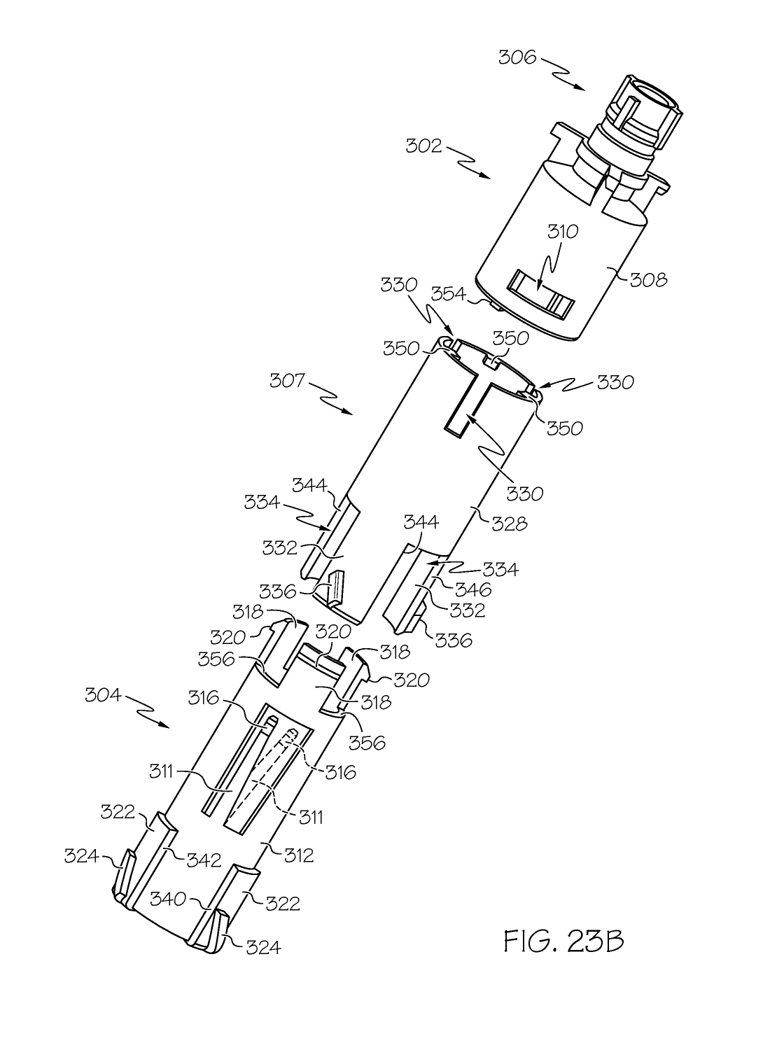

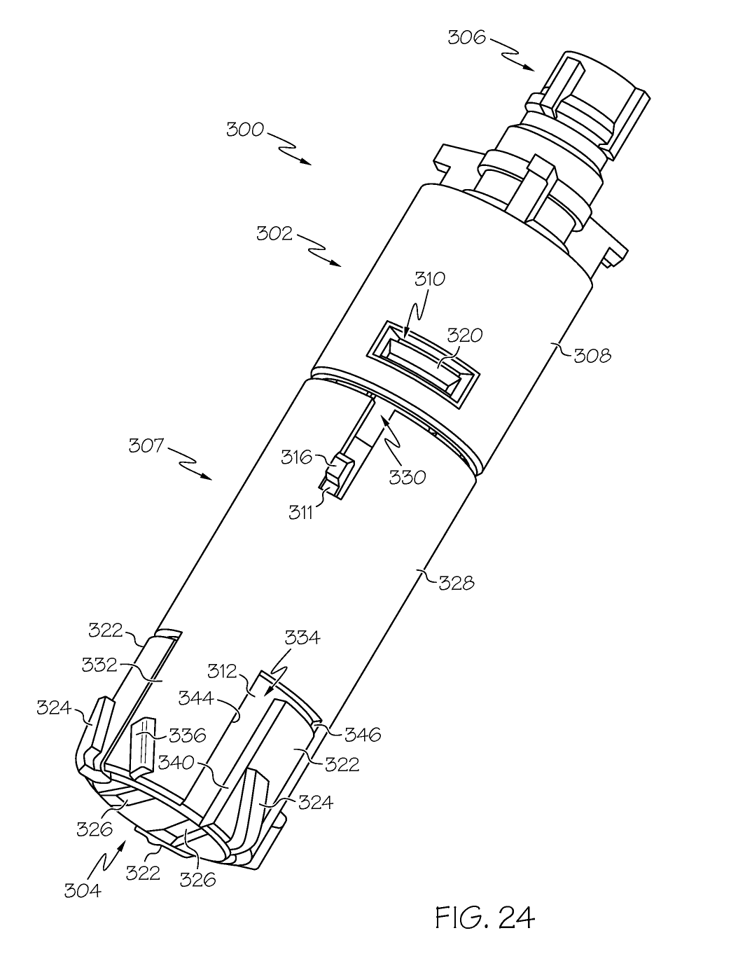

Referring now to FIGS. 23A, 23B and 24, a fourth embodiment of an end of dose signaling or notification mechanism 300 includes a connector lock 302, an end of dose click tube or signal part 307, and a connector 304. Connector lock 302 includes a button interfacing structure 306 carried by a tubular section 308 of connector lock 302. Tubular section 308 has a set of snap finger receiving windows 310 formed therethrough. In the illustrative example, three windows 310 are provided in tubular section 308 and each window 310 is generally rectangular in shape.

Connector 304 includes a main tubular portion 312 that has a generally rectangular spring arm receiving window 314 formed therethrough. A spring arm 311 is formed integrally with tubular portion 312 and extends generally axially upwardly into window 314. A knob or lug 316 is provided at the upper, free end of spring arm 311. A set of snap fingers 318 are formed integrally with portion 312 and extend axially upwardly from portion 312. In the illustrative example, three snap fingers 318 are provided. Each snap finger 318 includes a ramped ridge or flange 320 at its upper end. Flanges 320 of connector 304 are received in respective windows 310 of connector lock 302 when connector 304 and connector lock 302 are assembled together as shown in FIG. 24 (only one flange 320 and one window 310 are shown in FIG. 24).

Connector 304 has a set of pads 322 formed integrally with a lower end region of tubular portion 312. In the illustrative embodiment, there are three pads 322 that are spaced substantially equidistantly from each other about the circumference of tubular portion 312. Connector 304 also has external helical thread segments 324 that extend radially outwardly from respective pads 322 to engage complimentarily shaped helical grooves formed in another part (not shown) of the associated injection device such as a driver element (not shown) or housing (not shown). Connector 304 has internal helical thread segments 326 that extend radially inwardly from an internal surface of tubular portion 312 at the lower end region thereof Threads 326 engage complimentarily shaped helical grooves formed in another part (not shown) of the associated injection device such as a driver element (not shown).

Signal part 307 includes a tubular main portion 328 that has three straight, axially extending lug receiving slots 330 formed therethrough. Slots 330 are situated at the upper end of portion 328. Signal part 307 also has a set of arms 332 that are formed integrally with portion 328. Arms 332 extend axially from a bottom end of portion 328. In the illustrative example, there are three arms 332 that are spaced apart to define three pad receiving notches 334 at the lower end of signal part 307 as shown in FIGS. 23A and 23B. Signal part 307 has external helical thread segments 336 that each extend generally radially outwardly from the bottom end region of a respective arm 332 to engage a complimentarily shaped helical groove formed in another part (not shown) of the associated injection device such as a driver element (not shown) or housing (not shown).

End of dose signaling mechanism 300 is assembled by inserting connector 304 upwardly through the internal region or bore of signal part 307 so that snap fingers 318 extend beyond the upper end of signal part 307 and into the bore or interior region of connector lock 302. Receipt of flanges 320 in windows 310 securely fastens connector lock 302 and connector 304 together with signal part 307 being trapped between a lower annular edge 338 of connector lock 302 and pads 322 of connector 304 which are received in notches 334 of signal part 307.

In the illustrative example, the outer diameter of signal part 307 is substantially equal to the outer diameter of tubular section 308 of connector lock 302. Furthermore, when mechanism is assembled, lug 316 at the upper end of spring arm 311 is received in one of slots 330 of signal part 307 as shown in FIG. 24. By providing three slots 330 in signal part 307, there are three possible orientations that connector 304 may be inserted into signal part 307. Regardless of which slot 330 of the three slots 330 lug 316 occupies, the end of dose mechanism 300 will operate substantially the same.

Pads 322 each include an axial stop edge or surface 340 and an axial click edge or surface 342 as shown in FIG. 23 (edge 340 is visible on one of pads 322 and edge 342 is visible on another of pads 322 in FIGS. 23A and 23B). Notches 334 each are bounded by an axial stop edge or surface 344 and an axial click edge or surface 346. Edges 344, 346 are defined on opposite sides of each arm 332 of signal part 307. Edges 340, 342 of pads 322 and edges 344, 346 of arms 332 are each generally straight and extend generally parallel with one another.

Pads 322 are smaller in a circumferential direction of mechanism 300 than the respective notches 334 in which they are received. That is, an arc length between edges 340, 342 of each pad 322 is smaller than an arc length of each notch between edges 344, 346. Thus, when edges 342 of each pad abuts the corresponding edge 346 of a respective arm 332, a circumferential gap exists between edge 340 of each pad 322 and the respective edge 344 of the respective arm 332. These circumferential gaps define an amount by which signal part 307 is able to rotate about a main axis of mechanism 300 relative to connector 304 and connector lock 302. Thus, during dose setting of the injection device, signal part 307 is rotatable between a first rotational position in which edges 342 of pads 322 abut edges 346 of arms 332 and a second rotational position in which edges 342 of pads 322 are moved away from edges 346 of arms 332 and in which edges 340 of pads 322 are either closer to, or abut, edges 344 of arms 332.

In some embodiments, when signal part 307 is in the first rotational position shown in FIG. 24, spring arm 311 is unloaded and is in the solid line position shown in FIGS. 23A and 23B. In other embodiments, when signal part 307 is in the first rotational position, spring arm 311 is slightly flexed or tensed so as to be slightly loaded. As signal part 307 rotates from the first rotational position to the second rotational position, spring arm 311 flexes within window 314 to the dotted line position shown in FIGS. 23A and 23B. In the dotted line position, spring arm 311 is tensed or loaded by an increased amount as compared to the solid line position.

As was the case in the previous embodiments discussed above, during injection and after the button of the injection device has been pressed to its zero position, the internal pressure in the associated injection device results in a clamping force within the injection device that prevents rotation of signal part 307 from the second rotational position back toward the first rotational position. Signal part 307 is further held in the second rotational position before and during part of the injection cycle due to receipt of thread segments 336 in another part (not shown) of the injection device. Thus, after the button has been pressed to its zero position, signal part 307 remains in the second rotational position during injection until sufficient dissipation of the internal pressure of the injection device.

During dose setting and prior to the button reaching the zero position, the three thread segments 336 of signal part 336 are received in a narrow portion of a respective threaded groove of a six-start threaded part, similar to narrow portion 14a of part 3 of the first embodiment disclosed above (see FIGS. 13-17). The threaded grooves receiving the thread segments 336 have an enlarged space similar to enlarged space 14b of part 3. The thread segments 324 of connector 304 are received in the other three threaded grooves of the 6-start threaded part, but these three threaded grooves do not have any enlarged space. Thus, when thread segments 336 are situated in the enlarged space of the respective threaded groove of the 6-start threaded part, signal part 307 is able to rotate relative to connector 304 and connector lock 302. However, as explained above, signal part 307 does not start rotating from the second rotational position back toward the first rotational position until sufficient dissipation of the internal pressure of the injection device occurs.

As shown in FIG. 23B, single part 307 has three tabs 350 that project radially inwardly adjacent the upper end of main portion 328. As shown in FIG. 23A, connector lock 302 has three protrusions 352 that project radially inwardly adjacent the bottom end of tubular section 308. Connector lock also has three stop tabs 354 that are formed integrally with protrusions 352 and that extend axially beyond the bottom end of tubular section 308. As shown in FIG. 23B, connector 304 has three edges 356 at the top region of main tubular portion 312 that extend between snap fingers 318. During injection, the clamping force that inhibits rotation of signal part 307 from the second rotational position back to the first rotational position is created by tabs 350 being clamped between protrusions 352 of connector lock 302 and edges 356 of connector 304. Furthermore, when signal part 307 is in the second rotational position, tabs 350 abut stops 354 and when signal part 307 is in the first rotational position, tabs 350 are spaced from stops 354.

After the internal pressure in the injection device dissipates sufficiently, the clamping force acting on tabs 350 of signal part 307 is no longer strong enough to hold the signal part 307 against the force of the spring arm 311, and spring arm 311 moves from its relatively highly tensed or loaded position (e.g., the dotted line position of spring arm 311 in FIG. 23) back to the unloaded or slightly loaded position, as the case may be for a given embodiment (e.g., the solid line position of spring arm 311 in FIG. 23). As spring arm 311 moves in this manner, the torque imparted on signal part 307 by lug 316 drives signal part 307 to rotate relative to connector lock 302 and connector 304 from the second rotational position back to the first rotational position. When signal part 307 reaches the first rotational position, edges 346 of arms 332 of the signal part 307 will contact edges 342 of pads 322 of connector 304 resulting in both a tactile and an audible signal (e.g., a click), which informs the user that the injection has been fulfilled and the needle can be retracted from the skin. Thus, FIG. 24 corresponds to the end of dose condition of the injection device.

While the illustrative embodiment of mechanism 300 has pads 322 of connector 304 received in notches 334 of signal part 307, it should be appreciated that protrusions other than pads 322 and spaces other than notches 334 are within the scope of this disclosure. For example, one or more pockets or recesses in signal part 307 that do not extend all the way through signal part 307 would suffice in lieu of notches 334 in some embodiments. Also, one or more protrusions such as posts, fingers, lugs, ribs, and the like would suffice in lieu of pads 322 in some embodiments. As long as a surface or edge of signal part 307 moves into contact with a surface or edge of connector 304 upon signal part 307 returning back to the first rotational position under the urging of a suitable biasing element, such as spring arm 311, a suitable tactile or audible feedback will be produced within the associated injection device according to this disclosure.

In the illustrative example, spring arm 311 and window 314 are included as part of connector 304. In alternative embodiments, spring arm 311 and the associated window 314 are provided on an alternative connector lock 302. In such embodiments, the portion of connector lock carrying spring arm 311 is inserted into the bore of signal part 307. Alternatively or additionally, signal part 307 has grooves that receive lug 316 therein rather than slots 330 that extend all the way through main portion 328. Further alternatively or additionally, lug 316 is omitted from spring arm 311 and the signal part 307 has an inwardly extending protrusion that engages spring arm 311 to move it from the solid line position to the dotted line position. In such embodiments, slots 330 or grooves in signal part 307 are not needed.

Although certain illustrative embodiments have been described in detail above, many embodiments, variations and modifications are possible that are still within the scope and spirit of this disclosure as described herein and as defined in the following claims.

1. An injection device for injecting a medicament, the injection device comprising:
a housing,
a dose setting member movable relative to the housing for setting a dose to be injected, and
a signal part, wherein the signal part rotates about an axis relative to a surface within the injection device from a first rotational position to a second rotational position to increase loading on a spring when a dose is set due to rotation of the dose setting member relative to the housing, wherein an internal pressure builds up in the injection device during injection which results in the signal part being frictionally captured in the second rotational position between first and second internal parts of the injection device, and wherein, after the internal pressure dissipates by a sufficient amount during injection, the signal part rotates under the urging of the loaded spring from the second rotational position back to the first rotational position, a portion of the signal part moving into contact with the surface when the signal part reaches the first rotational position to produce tactile or audible feedback indicating that an end of dose condition has been reached.

2. The injection device of claim 1, wherein the spring comprises a torsion spring coupled to the signal part and to the first internal part.

3. The injection device of claim 2, further comprising a rotational tower having a track formed on an inner surface thereof, the track having an upper portion and a widened lower end, the signal part having a segment received in the track, wherein during rotation of the dose setting member to set the dose, the signal part moves axially relative to the rotational tower so that the segment moves from the widened lower end into the upper portion of the track and the loading of the torsion spring is increased due to relative rotation between the signal part and the first internal part as the signal part moves from the first rotational position to the second rotational position.

4. The injection device of claim 3, wherein, during injection, the segment moves into the widened lower end of the track and then, after internal pressure dissipation by a sufficient amount, the torsion spring loading decreases to move the segment of the signal part away from one side of the widened lower end toward another side that includes the surface as the signal part rotates from the second rotational position toward the first rotational position.

5. The injection device of claim 1, wherein the portion of the signal part that moves into contact with the surface to produce the tactile or audible feedback comprises an axially extending edge of the signal part.

6. The injection device of claim 5, wherein the surface contacted by the axially extending edge of the signal part comprises an axially extending edge of the second internal part.

7. The injection device of claim 1, wherein the signal part includes a main body having a first tab and the spring includes a spring arm of the signal part that extends from the main body in a curvilinear cantilevered manner such that the spring arm curves about the axis, the spring arm having a distal end with a second tab.

8. The injection device of claim 7, wherein the housing has a track formed on an inner surface thereof, the track having an elongated first segment and an enlarged space at an end of the first segment, wherein, after the setting of the dose and prior to injection of the dose, the first and second tabs are situated within the elongated first segment of the track and the spring arm is flexed due to the signal part having been rotated from the first rotational position to the second rotational position, and wherein, during the injection, the first and second tabs move into the enlarged space of the track, and when thereafter the internal pressure in the injection device has dissipated sufficiently, the spring arm deflects to spread the first and second tabs apart, thereby to move the signal part from the second rotational position back to the first rotational position.

9. The injection device of claim 7, further comprising a rotational tower located in an interior region of the housing, the rotational tower having a track formed on an inner surface thereof, the track having an elongated first segment and an enlarged space at an end of the first segment, wherein, after the setting of the dose and prior to the injection of the dose, the first and second tabs are situated within the elongated first segment of the track and the spring arm is flexed due to the signal part having been rotated from the first rotational position to the second rotational position, and wherein, during the injection, the first and second tabs move into the enlarged space of the track, and when thereafter the internal pressure in the injection device has dissipated sufficiently, the spring arm deflects to spread the first and second tabs apart, thereby to move the signal part from the second rotational position back to the first rotational position.

10. An end of dose notification mechanism for an injection device used for injecting a medicament, the end of dose notification mechanism comprising:
a rotational tower that is generally tubular and that has a track formed on an inner surface thereof, the track having an elongated first segment and an enlarged space at an end of the first segment,
a dose setting member movable relative to the rotational tower to set a dose to be injected, and
a signal part situated in an interior region of the rotational tower and movable along an axis defined by the rotational tower, the signal part having a main body with a first tab received in the track, the signal part having a spring arm cantilevered from the main body, the spring arm extending in a curved manner about the axis defined by the rotational tower, the spring arm having a distal end with a second tab,
wherein movement of the dose setting member to set the dose causes the main body of the signal part to rotate about the axis from a first rotational position to a second rotational position relative to the rotational tower such that the first tab is moved toward the second tab to increase loading of the spring arm,
wherein, after setting of the dose and prior to injection of the dose, the first and second tabs are situated within the elongated first segment of the track of the rotational tower and, during injection, the first and second tabs move into the enlarged space,
wherein an internal pressure builds up in the injection device during injection which results in the signal part being captured in the second rotational position between first and second internal parts of the injection device such that the first and second tabs are prevented from spreading apart within the enlarged space,
wherein, in response to the internal pressure dissipating by a sufficient amount, the spring arm deflects to spread the first and second tabs apart, thereby to rotate the main body of the signal part from the second rotational position back to the first rotational position to click the first tab against a surface of the rotational tower within the enlarged space of the track to signal the end of dose condition being reached.

11. The end of dose notification mechanism of claim 10, wherein the inner surface of the rotational tower is generally cylindrical and the elongated first segment forms a helical track along the inner surface.

12. The end of dose notification mechanism of claim 11, wherein the elongated first segment extends less than one revolution about the axis of the rotational tower.

13. The end of dose notification mechanism of claim 11, wherein the elongated first segment extends less than 180° about the axis of the rotational tower.

14. The end of dose notification mechanism of claim 10, wherein the elongated first segment extends along the inner surface of the rotational tower in substantially parallel relation with the axis of the rotational tower.

15. The end of dose notification mechanism of claim 14, wherein the rotational tower serves as an outer housing of the injection device.

16. The end of dose notification mechanism of claim 10, wherein the main body of the signal part is substantially cylindrical and wherein the signal part includes an annular flange extending radially inwardly from a top of the main body.

17. The end of dose notification mechanism of claim 16, wherein the annular flange is clamped between first and second internal parts of the injection device due to the internal pressure.

18. The end of dose notification mechanism of claim 17, wherein dissipation of the internal pressure results in the annular flange being unclamped from the first and second internal parts and permits the main body of the signal part to rotate about the axis of the rotational tower in response to deflection of the spring arm.

19. An end of dose mechanism for use with an injection device having at least two members that experience axial force when the injection device is operated to force medication from a cartridge of the injection device, the end of dose mechanism comprising
a spring, and
a signal part that rotationally moves from a first position to a second position during dose setting to increase loading of the spring, and a portion of the signal part being frictionally captured in the second position between surfaces of the at least two members due to internal pressure that builds up in the cartridge during injection, wherein after the internal pressure dissipates by a sufficient amount, the at least two members have released the portion of the signal part thereby to permit the signal part to rotate, under the urging of the spring, from the second position back to the first position, wherein the signal part has a first surface that contacts a second surface of the injection device to provide tactile or audible feedback indicating that an end of dose condition has been achieved.

20. The end of dose mechanism of claim 19, wherein the signal part includes a tab and the tab provides the first surface.

21. The end of dose mechanism of claim 19, wherein the signal part includes a notch that defines an axially extending edge and the axially extending edge provides the first surface.

22. The end of dose mechanism of claim 19, wherein the signal part includes a main body and the spring comprises a spring arm that extends from the main body in a cantilevered manner, the spring arm being curved about the axis.

23. The end of dose mechanism of claim 22, wherein the spring arm and the main body are integrally formed.

24. The end of dose mechanism of claim 19, wherein the spring comprises a torsion spring having a first end coupled to the signal part and having a second end coupled to one member of the at least two members.

25. An end of dose mechanism for use with an injection device, the end of dose mechanism comprising
a first part having a generally cylindrical portion with a window formed therethough, the first part having a spring arm formed integrally with the cylindrical portion and extending generally axially into the window, the first part having at least one protrusion that projects radially from the generally cylindrical portion, and
a signal part coupled to the first part for rotation between a first rotational position and a second rotational position, the signal part having a spring arm engaging portion, the signal part having at least one space that receives the at least one protrusion therein,
wherein during use of the injection device to set a dose, the signal part rotates from the first rotational position to the second rotational position and the spring arm engaging portion acts upon the spring arm to move the spring arm within the window to increase loading of the spring arm,
wherein during use of the injection device to inject a medication, an internal pressure builds up in the injection device during injection which results in the signal part being maintained in the second rotational position such that the spring arm is prevented from moving to decrease its loading,
wherein, in response to the internal pressure dissipating by a sufficient amount, the spring arm deflects and acts upon the spring arm engaging portion to rotate the signal part from the second rotational position back to the first rotational position to click an edge of the signal part against a surface of the protrusion received in the at least one space of the signal part.

26. The end of dose mechanism of claim 25, wherein a free end of the spring arm has a lug formed thereon and the spring arm engaging portion of the signal part comprises an edge defining a slot that receives the lug therein.

27. The end of dose mechanism of claim 25, wherein a thread segment is formed on the at least one protrusion.

28. The end of dose mechanism of claim 25, wherein at least one thread segment is formed on the signal part.

29. The end of dose mechanism of claim 28, wherein the signal part has at least one arm adjacent the at least one space and the at least one thread is formed on the at least one arm.

30. The end of dose mechanism of claim 25, wherein the at least one protrusion is situated adjacent a first end of the first part.

31. The end of dose mechanism of claim 30, wherein the first part has at least one snap finger that extends generally axially at a second end of the first part and the at least one snap finger has a ramped flange formed thereon.

32. The end of dose mechanism of claim 31, further comprising a second part having a window that receives the ramped flange therein to connect the first and second parts together.

33. The end of dose mechanism of claim 32, wherein the signal part is trapped between the at least one surface of the first part and an annular edge of the second part.

34. The injection device of claim 1, wherein the spring comprises a spring arm coupled to the first internal part and extending generally axially and wherein the signal part includes a spring arm engaging portion that engages a portion of the spring arm to increase loading on the spring arm as the signal part moves from the first rotational position to the second rotational position.

35. The injection device of claim 34, wherein a free end of the spring arm has a lug formed thereon and the spring arm engaging portion of the signal part comprises an edge defining a slot that receives the lug therein.
